(19) United States
(12) Patent Application Publication
(10) Pub. No.: US 2011/0263985 A1
(43) Pub. Date: Oct. 27, 2011

(54) Dual Path Color Doppler Imaging System and Method for Simultaneous Invasive Device Visualization and Vasculature Imaging

(75) Inventors: Thomas Gauthier, Seattle, WA (US); Aline Criton, Ventabren (FR); Matthew Bruce, Seattle, WA (US); Robert Entrekin, Kirkland, WA (US); Jeffry E. Powers, Bainbridge Island, WA (US)

(73) Assignee: KONINKLIJKE PHILIPS ELECTRONICS N.V., EINDHOVEN (NL)

(21) Appl. No.: 12/674,500

(22) PCT Filed: Aug. 14, 2008
(86) PCT No.: PCT/IB08/53270

§ 371 Date: Feb. 22, 2010

Related U.S. Application Data
(60) Provisional application No. 60/968,372, filed on Aug. 28, 2007.

(51) Int. Cl. A61B 8/06

(52) U.S. Cl. 600/454

(57) ABSTRACT

An ultrasound imaging system (10) is disclosed for creating simultaneous needle and vascular blood flow color Doppler imaging. A B mode image of an anatomical area of interest is created. A first set of Doppler image data optimized for the visualization of vascular blood flow is created along one Doppler image processing path. A second set of Doppler image data optimized for the visualization of a needle or other invasive device is created along another, parallel Doppler image processing path. A color Doppler image is created, and then displayed, by combining some or all of the B mode image, the first Doppler image data and the second Doppler image data based on a plurality of user selectable modes.

This invention relates to systems and methods for creating color

Doppler images on an ultrasound imaging system and more particularly for creating color Doppler images using two separate color Doppler processing modes optimized for imaging tissue or invasive (interventional) medical devices such as needles.

Ultrasound imaging is commonly used to image the insertion, use or operation of invasive medical devices and instruments within the body. For example, fine needle aspiration (FNA), core biopsy, radio-frequency ablation (RFA), or percutaneous ethanol injection (PEI) are all procedures that require insertion of an invasive device into the patient. When performing, for example, a radio-frequency ablation, a doctor must be able to visualize the target (e.g. the hepatocellular carcinoma to be ablated), the needle approaching the target and any vasculature surrounding the target. Imaging of the vasculature is key for ensuring that no major vessel is punctured during needle insertion as well as ensuring no other hemorrhaging has occurred.

Currently, physicians visualize the target with grayscale imaging (B mode) and the vasculature using color Doppler (colorflow) imaging. Colorflow images are a composite of a B-mode (grayscale) image with the flow overlaid as a color Doppler image. The B mode image shows the tissue structure and other stationary objects and tissues in a region being examined. The color Doppler image is formed by acquiring ensembles of Doppler data over time along each line in the image, estimating the Doppler shift using an ensemble of data at each point along the line, and forming a color image of the vasculature where the color for each point along the line depends on the velocity of the flow of the sample volume at that point. In this way the flow of blood, displayed in the Doppler mode, is functionally depicted in color in the surrounding tissue and blood vessels structurally shown in the B-mode image. Typically, B-mode imaging is also used to image the invasive device. In principle, color Doppler imaging could also be used to image the invasive device as it approaches the target, and at least one commentator has suggested doing so. In practice, however, the color Doppler settings that are required to effectively image the blood flow in vasculature are very different from those used to better visualize the slow moving needle. The combination of B-mode and color Doppler imaging in prior art ultrasound imaging devices is, therefore, only capable of allowing physicians to effectively visualize either the needle or the vascular flow.

There is therefore a need for an ultrasound imaging system that permits simultaneous and effective visualization of both the vascular flow and an invasive device with Doppler.

In accordance with the principles of the present invention an ultrasound system is provided for imaging an invasive device during an invasive procedure. Blood flow and the invasive device are imaged using Doppler modes with different settings, one optimized for blood flow and the other optimized to visualize the invasive device. The different Doppler modes could be velocity imaging for the blood flow and power Doppler for the invasive device, for example. Another embodiment would use colorflow Doppler or power Doppler for imaging both the flow and the invasive device, but with different color maps.

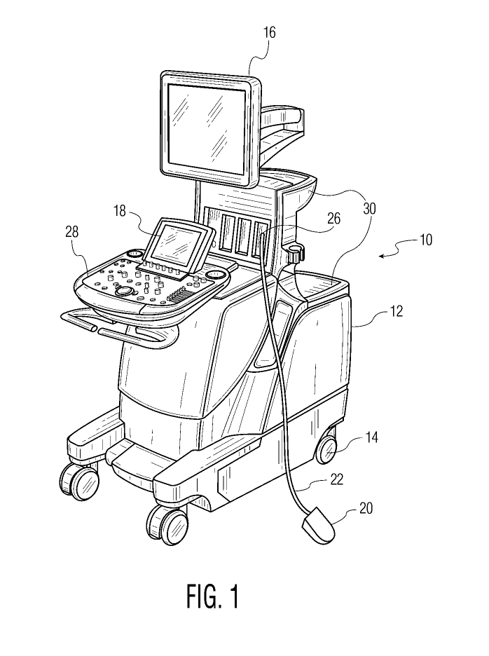

FIG. 1 is a perspective view of an ultrasound imaging system according to one example of the invention.

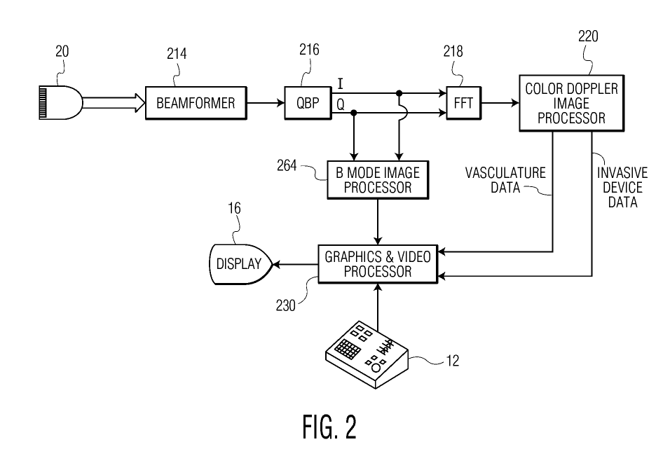

FIG. 2 is a block diagram of an ultrasound imaging system constructed in accordance with the principles of the present invention.

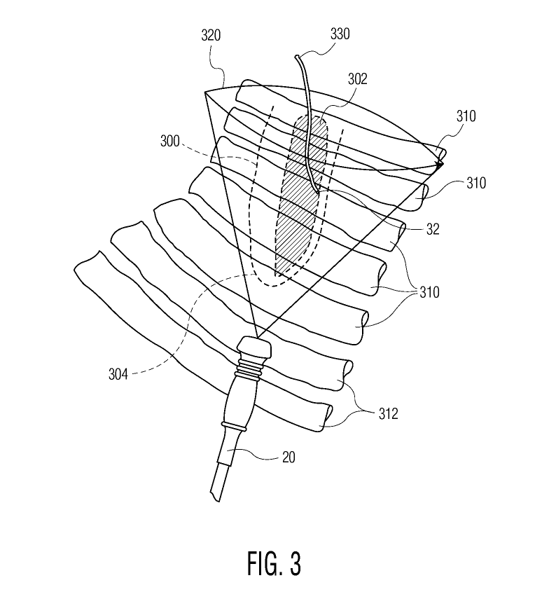

FIG. 3 is a schematic diagram illustrating the ultrasonic imaging of an invasive device in the heart by a transthoracic transducer probe.

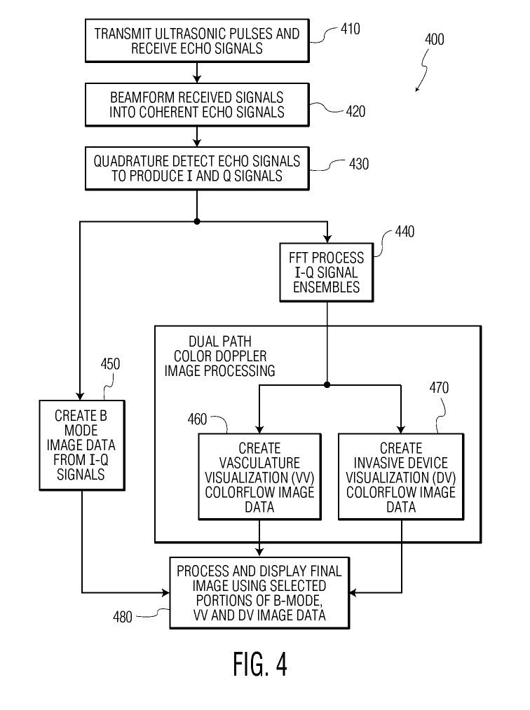

FIG. 4 is a flow chart depicting a process flow diagram of a dual path color Doppler processing method in accordance with an embodiment of the invention.

An ultrasound imaging system 10 according to one example of the invention is illustrated FIG. 1. The system 10 includes a chassis 12 containing most of the electronic circuitry for the system 10. The chassis 12 may be mounted on a cart 14, and a display 16 is mounted on the chassis 12. An imaging probe 20 may be connected through a cable 22 to one of three connectors 26 on the chassis 12. The chassis 12 includes a keyboard and user controls, generally indicated by reference numeral 28, for allowing a sonographer to operate the ultrasound system 10 and enter information about the patient or the type of examination that is being conducted. At the back of the control panel 28 is a touchscreen display 18 on which programmable softkeys may be displayed for supplementing the keyboard and controls 28 in controlling the operation of the system 10. The chassis 12 generally also includes a pointing device such as a trackball that may be used to, for example, manipulate an on-screen pointer. The chassis 12 may also include one or more buttons (not shown) which may be pressed or clicked after manipulating the on-screen pointer. These operations are analogous to a mouse being used with a computer.

In operation, the imaging probe 20 is placed against the skin of a patient (not shown) and held stationary to acquire an image of blood or tissue in a two or three dimensional region beneath the skin. The image is presented on the display 16, and it may be recorded by a recorder (not shown) placed on one of the two accessory shelves 30. The system 10 may also record or print a report containing text and images. Data corresponding to the image may also be downloaded through a suitable data link, such as the Internet or a local area network.

One example of the electrical components of the ultrasound imaging system 10 is illustrated in FIG. 2. Ultrasonic signals are transmitted by the transducer array 20 of an ultrasound probe and the resultant echoes received by the elements of the transducer array. The echo signals received by the elements of the array are formed into a single signal or beam by a beamformer 214. The echo signal information is detected as I and Q signal components by a quadrature bandpass filer (QBP) 216, which produces quadrature I and Q signal components. QBP filters are described in detail in U.S. Pat. No. 6,050,942, which is incorporated herein by reference. A number of such signal components from the sites in the body (sample volumes) being diagnosed are acquired over time at an ensemble pulse repetition frequency (PRF) and are applied to a fast Fourier transform (FFT) processor 218, which estimates the Doppler frequency shift at the sample volume locations. In accordance with the principles of the present invention, this basic Doppler data is post processed by a dual path color Doppler image processor 220, which, as will be discussed in more detail below, further refines the data by techniques such as wall filtering and/or signal segmentation to create a color Doppler image. Conceptually, the dual path color Doppler image processor 220 processes the Doppler data along two independent paths each with its own settings and optimizations. The dual path color Doppler image processor 220 therefore produces two sets of data. As will be discussed in more detail below, the settings and optimizations of one path of the image processor 220 produce image data suitable for optimal visualization of the blood flow in the vasculature while the other path produces image data most suited for visualizing invasive devices. However, it should be understood that other settings and optimizations are possible for each path as may be required to produce images suitable for other types of anatomy or devices being visualized.

Intermittently during the reception of Doppler echoes, B-mode echoes may be received. These echoes may also be processed into I and Q signal components are then amplitude detected by taking the square root of the sum of the squares of the I and Q values in a B mode image processor 264. The B-mode and color Doppler image data is received by a graphics and video processor 230 where they are converted to image data and then coordinated and overlaid in the desired display format such as a sector or rectilinear image. Graphics such as textual patient information may also be overlaid on the image display. From the combined image data, the graphics and video processor 230 produces video drive signals compatible with the requirements of the display 16.

FIG. 3 illustrates the ultrasonic imaging of an invasive device 330 in the heart by a transthoracic transducer probe 20. In this example, a heart 300 is located behind the left side of a rib cage (shown in partial outline behind the rib cage 310, 312). Outlined within the heart and cross-hatched is the left ventricle 302 of the heart 300. The left ventricle can be accessed for ultrasonic imaging by scanning the heart from between the ribs 310, 312 for adult patients and, for some pediatric patients, by scanning upward from below the lowest rib 312. The probe 20 scans the heart from a heart apex 304 as indicated by an outline 320 of the field of view scanned by the probe 20. As FIG. 3 illustrates, the left ventricle 302 can be fully encompassed and scanned by the field of view 320 directed from between the rib cage 310, 312.

Also shown in FIG. 3 is an invasive device 330, which performs a function within the body. In this drawing, the invasive device is shown as a catheter. It could, however, also be some other tool or instrument such as a needle, a surgical tool such as a dissection instrument or stapler or a stent delivery, electrophysiology, or balloon catheter, a therapy device such as a high intensity ultrasound probe or a pacemaker or defibrillator lead, a diagnostic or measurement device such as an IVUS or optical catheter or sensor, or any other device which is manipulated and/or operates within the body. Just as with the ablation example discussed above, insertion and manipulation of the catheter must be carefully monitored and visualized to prevent unwanted injury or trauma to the patient.

While FIG. 3 illustrates scanning of the region 320 in a conical, three dimensional field of view, one skilled in the art will recognize that other scan formats may also be employed, such as those that scan a rectangular or hexagonal pyramidal field of view or a two dimensional image plane. It will also be appreciated that probes other than transthoracic probes may be used for scanning such as transesophageal probes, intracavity probes such as vaginal or rectal probes, and intravascular probes such as catheter-mounted transducer probes. While an electronically scanned two-dimensional array transducer will generally be preferred for three dimensional imaging, mechanically scanned arrays may be preferred for some 3D applications, such as abdominal procedures.

FIG. 4 is a process flow diagram of a dual path color Doppler processing method in accordance with an embodiment of the invention. At step 410, a transducer array transmits ultrasonic pulses into a patient, and receives echo signals from ultrasonic energy reflected by the patient's blood, organs and other tissue. These echo signals are typically processed by a beamformer into coherent echo signals as depicted at step 420. At step 430, a QBP filter is used to produce quadrature I and Q samples of the echo signals. These signal samples are in turn used at steps 440 and 450. B mode image data is created from the I-Q signals at step 450. The I and Q signal samples are further processed by, for example, an FFT processor to produce Doppler frequency shift estimates as shown at step 440. These Doppler signals are in turn directed along dual paths to steps 460 and 470 for creating colorflow image data optimized for flow visualization and invasive device visualization, respectively.

As discussed above, Doppler ultrasound works by detecting a frequency shift in the returned echo signals compared to the frequency of the signals applied to the body. Such a frequency shift can be detected through spectral analysis of the returned echo signals using a fast Fourier transform (FFT) or equivalent signal processing technique. Colorflow image data is created from the results of such analysis since the frequency shift is proportional to velocity and typically, each point in the color image formed from that data will reflect the average velocity, or other measured attribute such as flow variance, of the sample volume flow at that point.

When imaging and visualizing vasculature, the colorflow image data is created at step 460 using settings that suitable for effective visualization of blood flow. Effective visualization of the blood flow of vasculature requires the detection and processing of the low level echo signals returned from flowing blood. In particular, visualizing blood flow requires the detection and processing of the high-frequency content of the Doppler ensembles because that frequency content is proportional to the velocity of the blood flow. In addition, echoes from nearby sample volumes may contain low frequency, high intensity artifacts that are typically caused by moving muscle or artery walls. These artifacts interfere with the ability of the physician to clearly visualize the flow conditions.

In an effort to mitigate the effects of such artifacts, methods for signal segmentation have been developed. Signal segmentation is the process of separating and differentiating signals from one another based on one or more measurable criteria. The traditional way of removing clutter from a color Doppler image of vascular blood flow is with a wall filter. A wall filter is designed to exclude Doppler signals with low frequencies such as those returned from a vessel wall or invasive device. Such a filter consists of a high-pass or band pass filter with suitable cutoffs for excluding the low frequency signals. Thus, the wall filter differentiates between signals based on the frequency of those signals.

Another means of signal segmentation takes advantage of the fact that moving tissue and invasive devices return echo signals with a higher amplitude than echoes returned by blood cells. Most invasive devices being imaged will produce a high amplitude echo signal. Thus, to effectively visualize nearby or surrounding blood flow, high amplitude signals caused by the presence of an invasive device should be removed. On the other hand, effectively visualizing an invasive device would require selecting such signals, instead of rejecting them.

In accordance with the principles of the present invention, effective simultaneous vascular blood flow visualization and invasive device visualization at steps 460 and 470, respectively, each require their own processing characteristics, settings and optimizations. For example, step 460 could employ a high pass wall filter for blood flow visualization which would reject lower frequencies of both the invasive device and moving tissue. Correspondingly, step 470 could employ a lower frequency band pass wall filter to reject higher frequency blood flow signals and stationary tissue clutter. As another example, the two steps could use different color maps, with the motion of the invasive device mapped to a completely different range of colors than those of the blood flow. As yet another example, signal amplitude segmentation could be employed to compare the received echo signals to a threshold. Lower amplitude signals from blood would be processed for blood flow visualization in step 460 while higher amplitude signals would be processed for invasive device visualization in step 470 with stationary clutter removed. Still another optimization is to employ different FFT processing for steps 460 and 470. Since the low frequency motion of an invasive instrument need be sampled with a lower PRF (the samples are separated by greater time intervals) than higher frequency blood flow, samples could be omitted for the invasive device FFT resulting in ensembles with samples more widely spaced in time than those used by the blood flow FFT. Thus, different FFT processing is used for steps 460 and 470, with the FFT step 440 implemented differently for each of the blood flow and invasive device visualization steps. One skilled in the art will recognize that a steering circuit can be employed at the inputs to steps 460 and 470 to steer I and Q or Doppler signals to the process appropriate for the characteristics of each signal. Such a steering circuit effectively provides a degree of signal segmentation.

B mode, blood flow optimized, and invasive device optimized image data is combined at step 480 to produce and display the final image. In the illustrated embodiment, the final image is rendered based on one or more modes selected by the user. In one mode, step 480 might display the B mode tissue image overlaid with the color Doppler flow image only. Alternatively and in response to selection of another mode by the user, the final image that is displayed may contain the B mode image overlaid with the color Doppler invasive device image only.

On the basis of selection of a third mode, the final image may be some combination of all three types of image data: the B mode tissue image overlaid with color Doppler blood flow image further overlaid with the color Doppler invasive device image. As previously mentioned, one possibility when operating in this mode, to better differentiate blood flow from the invasive device, two different color maps may be used to render the motion of the invasive device in its own distinctive color. For example, a range of reds and blues may be used for mapping the image of the blood whereas a single color map such as yellow may suffice for displaying the invasive device. An adjustable user control for the color mapping, wall filter cutoffs, and/or intensity thresholding would enable the user to adjust the segmentation for the extant signal conditions.

One skilled in the art will recognize that the principles of the present invention may be applied to imaging in other Doppler modes in addition to or alternatively to colorflow (velocity) mode, such as power Doppler (Doppler intensity) mode.

Although the invention has been described with reference to the disclosed examples, persons skilled in the art will recognize that changes may be made in form and detail without departing from the spirit and scope of the invention. Such modifications may be well within the skill of those ordinarily skilled in the art. Accordingly, the invention is not limited except as by the appended claims.

What is claimed is:

1. A method for creating a color Doppler image of an image field including blood flow and an invasive device on an ultrasound imaging system, comprising:
transmitting an ensemble of ultrasound pulses down at least one line of sight in an image field;
receiving echo signals from the at least one line of sight;
Doppler processing the echo signals using a first setting to create color image data of blood flow along the at least one line of sight;
Doppler processing the echo signals using a second setting to create color image data of an invasive device along the at least one line of sight; and
creating the color Doppler image by selectively combining the color image data of blood flow with the color image data of the invasive device.

2. The method of claim 1 wherein creating a color Doppler Image further comprises:
forming a B mode image based on the amplitude of received echoes; and
creating the color Doppler image by selectively combining the color image data of blood flow, the color image data of the invasive device, and the B mode image.

3. The method of claim 2 wherein selectively combining comprises combining some, all or none of each type of image data.

4. The method of claim 1 wherein Doppler processing the echo signals to create color image data of blood flow comprises Doppler processing the echo signals to optimize the visualization of vascular flow.

5. The method of claim 4 wherein Doppler processing the echo signals to optimize the visualization of vascular flow comprises processing Doppler signals to filter at least one of high frequency signal content and low amplitude signal content.

6. The method of claim 1 wherein Doppler processing the echo signals to create color image data of an invasive device comprises Doppler processing the echo signals to optimize the visualization of an invasive device.

7. The method of claim 6 wherein Doppler processing the echo signals to optimize the visualization of an invasive device comprises processing Doppler signals to filter at least one of: low frequency signal content and high amplitude signal content.

8. The method of claim 1 wherein the color image data of blood flow and the color image data of an invasive device are each created using different color maps.

9. An ultrasound imaging system comprising:
a display;
a processor coupled to the display;
a user interface coupled to the processor;
a transducer coupled to the processor and operable to transmit a plurality of ultrasound pulses down at least one line of sight in an image plane or volume and receive echoes in response to the pulses; and
wherein the processor is operable to Doppler process the echoes with a first setting to create color image data of vascular blood flow along the at least one line of sight;
wherein the processor is further operable to Doppler process the echoes with a second setting to create color image data of an invasive device along the at least one line of sight; and
wherein the processor is further operable to create a color Doppler image by selectively combining the color image data of vascular blood flow with the color image data of the invasive device,
wherein the user interface is operable to vary at least one of the first or second settings.

10. The ultrasound imaging system of claim 9 wherein the processor is further operable to:
form a grayscale image from received echo signals; and
create the color Doppler image by selectively combining the color image data of vascular blood flow, the color image data of the invasive device, and the grayscale image.

11. The ultrasound imaging system of claim 9 wherein selectively combining comprises combining some, all or none of each image data.

12. The ultrasound imaging system of claim 9 wherein Doppler processing the echoes to create color image data of vascular blood flow along the at least one line comprises Doppler processing the echoes to optimize the visualization of vascular blood flow.

13. The ultrasound imaging system of claim 12 wherein Doppler processing the echoes to optimize the visualization of vascular blood flow comprises filtering Doppler signals to select at least one of: high frequency signal content and low amplitude signal content.

14. The ultrasound imaging system of claim 9 wherein Doppler processing the echoes to create color image data of an invasive device along the at least one line comprises Doppler processing the echoes to optimize the visualization of an invasive device.

15. The ultrasound imaging system of claim 14 wherein Doppler processing the echoes to optimize the visualization of an invasive device comprises filtering Doppler signals to select at least one of: low frequency signal content and high amplitude signal content.

16. The ultrasound imaging system of claim 9 wherein the color image data of vascular blood flow and the color image data of an invasive device are each created using a different color map.

17. The ultrasound imaging system of claim 9 wherein the color image data of vascular blood flow and the color image data of an invasive device are each created using a different ensemble of echo signals.
